(19) United States
(12) Patent Application Publication
(10) Pub. No.: US 2008/0286165 A1
(43) Pub. Date: Nov. 20, 2008

(54) Guanidine Based Composition and System for Same

(76) Inventors: Robert K. Graupner, Hillsboro, OR (US); J. Dustin Hultine, Portland, OR (US); James Alden Van Vechten, Lake Oswego, OR (US)

Correspondence Address: BROOKS KUSHMAN P.C., 1000 TOWN CENTER, TWENTY-SECOND FLOOR, SOUTHFIELD, MI, 48075, US

(21) Appl. No.: 11/568,629

(22) PCT Filed: May 3, 2005
(86) PCT No.: PCT/US05/15920

§ 371 Date: Nov. 3, 2006

Related U.S. Application Data
(60) Provisional application No. 60/568,551, filed on May 5, 2004.
(60) Provisional application No. 60/572,677, filed on May 20, 2004.
(60) Provisional application No. 60/579,904, filed on Jun. 15, 2004.
(60) Provisional application No. 60/584,707, filed on Jun. 30, 2004.

(51) Int. Cl. C01C 1/00; C07C 277/00; C10L 3/00

(52) U.S. Cl. 422/148; 564/241; 48/197 FM

(57) ABSTRACT

A method and apparatus for generating energy from a composition containing guanidine and a method for providing the composition containing guanidine. The apparatus includes a container such as tank (1) for providing the composition, and a container such as tank (2) for providing water. The composition is delivered from tank (1) to a container such as reactor (3) for reacting the guadinine composition with water, supplied from tank (2), to form ammonia. The apparatus may also include buffer tank (4) for storing the ammonia produced by the reactor. The ammonia produced from the reactor of the guadinine composition with water is delivered from the buffer tank (4) to a container such as chamber (5) for oxidizing ammonia to form water and nitrogen generating energy

### FIELD OF INVENTION

The present invention relates to a guanidine based composition. The present invention also relates to a method for generating energy from a guanidine based composition. The present invention also relates to a method for producing the guanidine based composition.

### BACKGROUND OF INVENTION

Fossil fuels constitute the largest source of energy supply in the world. Their availability in large quantities, high energy density, and relatively low cost makes fossil fuels the fuel of choice for many applications for both industries and consumers. Nonetheless, an alternative to fossil fuels as an energy source is highly desirable for safety reasons, for environmental reasons, and to reduce the dependence of the industrialized world on oil imported from relatively few oil-producing countries. In spite of these compelling arguments in favor of new energy sources, no economical, safe and readily available fuel has been found to be a viable alternative for fossil fuels, especially in automotive and portable power applications, even though extensive research has been aimed at developing a fuel having the many positive characteristics of gasoline without its drawbacks.

A new, alternative fuel should fulfill several requirements. The cost of the fuel should be competitive with the cost of current energy sources. The fuel should have no undesirable emissions. It should have a high energy density, as gasoline does, to avoid the need for frequent refilling. The fuel should be easy to handle safely. Preferably, it should be non-explosive and of low toxicity. Finally, it should be available through a distribution infrastructure that can be readily expanded. There is therefore a need in the art for a fuel having all of the above characteristics.

Current efforts directed to new fuels have not provided a fuel that is cost effective compared to gasoline. Furthermore, the effectiveness of any of the proposed alternatives as a source of energy appears problematic. For example, although hydrogen may be obtained from fossil or non-fossil sources, problems related to handling during transportation and to storage make hydrogen a poor choice for a consumer-oriented fuel. Other fuels, such as methanol, are toxic and flammable and still require a certain amount of fossil fuel, in the form of either natural gas or carbon monoxide, to be economically produced. Sources of hydrogen such as chemical hydrides, while easy and effective to use, require a recycle loop if it is desired to re-utilize the hydrides, as discussed in U.S. Pat. Nos. 5,804,329 and 6,534,033.

Amendola (U.S. Pat. App. 20030219371) proposed methods for production of the fuels ammonia and hydrogen from compositions containing urea. Amendola used the term “urea” to denote any one of the components of commercial urea, including urea, NH₂CONH₂, ammonium carbamate, ammonium carbonate, ammonium bicarbonate, ammonium formate, ammonium acetate, or a mixture of two or more of these components. Urea as a carrier of energy is limited in application by the solubility and melting point of urea. The melting point of urea (132-135° C.) is too high to allow convenient handling of the solid composition in molten form. The solubility of urea is approximately 1 kg of urea in 1 kg of water. When a saturated solution of urea in water is converted to ammonia, approximately 1 volume of water vapor and one-half volume of carbon dioxide will be produced for every volume of ammonia produced, making the product unsatisfactory for immediate use as a fuel. Also, water vapor, carbon dioxide and ammonia spontaneously combine to form the solid ammonium carbonate which can deposit on the interior surfaces of the apparatus, impeding proper operation. The necessity to carry over three times the amount of water needed for the reaction of urea to form ammonia substantially increases the weight of the vehicle, adversely affecting fuel economy.

The problems encountered with urea compositions can be avoided by the use of other energy carriers which can react with water to form ammonia. For example, free-base guanidine (CN₃H₅) can be decomposed into ammonia and carbon dioxide in the presence of water. Guanidine is infinitely soluble in water, and has a convenient melting point (50° C.) allowing it to be transported as a solid, yet handled within an engine as a liquid. As a highly concentrated solution in water or as a melt, guanidine can be combined with the exact amount of water required for complete reaction, thereby minimizing the amount of water vapor in the ammonia product. Upon reaction to form ammonia, a one-third volume of carbon dioxide will be produced for every volume of ammonia, allowing the product to be immediately used as a fuel. The low levels of water present in the product will minimize the creation of ammonium carbonate deposition within the apparatus. Guanidine has an energy density approximately 1.5 times higher than a dry urea composition. The energy content of 2 tonnes of guanidine is equivalent to about the energy content of 3 tonnes of urea, thereby reducing transportation and storage costs for the dry materials used in the fuel.

Guanidine can be made from ammonia and urea (Shaver, U.S. Pat. No. 3,108,999), both of which are currently made in very large quantities. According to U.S. Pat. No. 4,157,348, commercially produced urea contains total guanidine (the total of free-base guanidine, all guanidine salts, and guanidine derivatives such as guanylurea) at levels below 1% w/w which can be extracted to provide another source of the fuel. The guanidine in commercial urea is likely to be in the form of salts such as guanidine carbonate and guanidine hydroxide given the high reactivity of free-base guanidine with the carbon dioxide and water present in the urea production process. The guanidine salts recovered from urea production can be partially converted into free-base guanidine by reaction with a strong base. Guanidine fuel can be produced in a sustainable manner using the Shaver process because the raw materials are hydrogen, nitrogen, and carbon dioxide. Nitrogen can be extracted from the air at low cost. Carbon dioxide is readily available from fossil fuel combustion and other sources. The hydrogen can be produced by electrolysis of water using electricity produced from wind turbines at remote sites, such as the Aleutian Islands or parts of North Dakota, Oregon, and Washington. Using wind power or other sustainable energy sources, the guanidine can be produced and delivered at a price which can allow it to be competitive with gasoline or diesel fuel.

Guanidine is a solid at room temperature, and can be distributed in disposable or recyclable containers at retail outlets, such as grocery stores. Guanidine is also a fertilizer with about a 71% by weight nitrogen content, which would allow agricultural operations, located near sources of wind power, to become completely sustainable both in the use of synthetic fertilizers and for the fuel needed to operate the extensive power-equipment used in modern farming.

### BRIEF SUMMARY OF THE INVENTION

Accordingly, it is an object of the invention to provide a method for generating energy from water and a composition containing guanidine. The method in a first embodiment comprises: (a) reacting the composition with water to form ammonia; and (b) oxidizing the ammonia formed in step (a) to form water and nitrogen generating energy.

The method in a second embodiment comprises: (a) reacting the composition with water to form ammonia; (b) converting the ammonia formed in step (a) into nitrogen and hydrogen; and (c) oxidizing the hydrogen formed in step (b) to form water generating energy.

It is another object of the invention to provide an apparatus for generating energy from a composition containing guanidine and water. The apparatus in a first embodiment comprises: (a) a first container for providing the composition; (b) a second container for providing water; (c) a third container for reacting the composition with water to form ammonia, wherein the third container is connected to the first container by means for delivering the composition from the first container to the third container, and the third container is connected to the second container by means for delivering water from the second container to the third container; (d) a fourth container for storing ammonia, wherein the fourth container is connected to the third container by means for delivering ammonia from the third container to the fourth container; and (e) a fifth container for oxidizing ammonia to form water and nitrogen generating energy, wherein the fifth container is connected to the fourth container by means for delivering ammonia from the fourth container to the fifth container.

The apparatus in a second embodiment comprises: (a) a first container for providing the composition; (b) a second container for providing water; (c) a third container for reacting the composition with water to form ammonia, wherein the third container is connected to the first container by means for delivering the composition from the first container to the third container, and the third container is connected to the second container by means for delivering water from the second container to the third container; (d) a fourth container for storing ammonia, wherein the fourth container is connected to the third container by means for delivering ammonia from the third container to the fourth container; (e) a fifth container for converting ammonia into nitrogen and hydrogen, wherein the fifth container is connected to the fourth container by means for delivering ammonia from the fourth container to the fifth container; (f) a sixth container for providing hydrogen, wherein the sixth container is connected to the fifth container by means for delivering hydrogen from the fifth container to the sixth container; and (g) a seventh container for oxidizing hydrogen to form water generating energy, wherein the seventh container is connected to the sixth container by means for delivering hydrogen from the sixth container to the seventh container.

The apparatus in a third embodiment comprises: (a) a first container for providing the composition; (b) a second container providing water; (c) a third container for reacting the composition with water to form ammonia, wherein the third container is connected to the first container by means for delivering the composition from the first container to the third container and the third container is connected to the second container by means for delivering water from the second container to the third container; (d) a fourth container for storing ammonia, wherein the fourth container is connected to the third container by means for delivering ammonia from the third container to the fourth container; and (e) a fifth container for converting ammonia to nitrogen and hydrogen, wherein the fifth container is connected to the fourth container by means for delivering ammonia from the fourth container to the fifth container; (e) a sixth container for storing hydrogen, wherein the sixth container is connected to the fifth container by means for delivering hydrogen from the fifth container to the sixth container; and (f) a means for delivering hydrogen from the sixth container to a hydrogen storage tank in a vehicle which uses hydrogen as a fuel to generate energy.

It is another object of the invention to provide a method for providing a guanidine containing composition. The method in one embodiment comprising: (a) using a source of energy to separate hydrogen from water; (b) reacting the hydrogen formed in step (a) with nitrogen to form ammonia; (c) reacting a portion of the ammonia formed in step (b) with carbon dioxide to form urea; (d) reacting the urea formed in step (c) with a portion of the ammonia formed in step (b) to form a composition containing guanidine.

It is another object of the invention to provide an apparatus for providing a guanidine containing composition. The apparatus in one embodiment comprising: (a) a first container for separating hydrogen from water using energy; (b) a second container for providing nitrogen; (c) a third container for reacting hydrogen with nitrogen to form ammonia; wherein the third container is connected to the first container by means for delivering hydrogen from the first container to the second container and the third container is connected to the second container by means for delivering nitrogen from the second container to the third container; (d) a fourth container providing carbon dioxide; (e) a fifth container for reacting ammonia with carbon dioxide to form urea; wherein the fifth container is connected to the third container by means for delivering ammonia from the third container to the fifth container and the fifth container is connected to the fourth container by means for delivering the carbon dioxide from the fourth container to the fifth container; (f) a sixth container for reacting urea ammonia to form a composition containing guanidine; wherein the sixth container is connected to the third container by means for delivering ammonia from the third container to the sixth container, and the sixth container is connected to the fifth container by means for delivering urea from the fifth container to the sixth container.

As is further discussed below, the composition containing guanidine when combined with water represents a suitable alternative to fossil fuels. The composition has a high specific energy and energy density, is environmentally friendly and easy to handle. Guanidine has been demonstrated to be a fertilizer, so spills of guanidine would present a small environmental hazard.

### BRIEF DESCRIPTION OF THE DRAWINGS

FIG. 1 shows a schematic description of encapsulated guanidine composition

FIG. 2 shows a schematic description of a first embodiment of the apparatus of the invention.

FIG. 3 shows a schematic description of a second embodiment of the apparatus of the invention.

FIG. 4 shows a schematic description of a third embodiment of the apparatus of the invention.

FIG. 5 shows a schematic description of a fourth embodiment of the apparatus of the invention.

FIG. 6 shows a schematic description of an embodiment of the reaction chamber with an H₂ selective semi-permeable membrane.

FIG. 7 shows a schematic description of a fifth embodiment of the apparatus of the invention.

FIG. 8 shows a schematic description of an embodiment of the reaction chamber with a replaceable cartridge, electrical heater and heat pipe array.

FIG. 9 shows a schematic description of an embodiment of the apparatus for providing a guanidine containing composition.

### DRAWINGS—REFERENCE NUMERALS

1. Container providing guanidine composition
2. Container providing water
3. Container for reacting composition with water to form ammonia
4. Container for storing ammonia
5. Container for oxidizing ammonia—as Internal Combustion Engine (ICE) or Fuel Cell (FC)
6. Container for converting ammonia to nitrogen and hydrogen
7. Container for storing hydrogen
8. Container for oxidizing hydrogen to form water generating energy
9. Exhaust water condenser
10. Container for Selective Catalytic Reduction (SCR)
11. Nitrogen oxide free exhaust
12. Exhaust
13. Guanidine composition
14. Encapsulating layer
21. Container for electrolysis of water or brine
22. Container for storing nitrogen
23. Container for converting nitrogen and hydrogen into ammonia
24. Container for storing carbon dioxide
25. Container for converting ammonia and carbon dioxide into urea
26. Container for converting urea and ammonia into guanidine composition
27. Container for storing guanidine composition
60. Means for delivering gaseous substances
63. Means for delivering liquid substances
68. Means for delivering guanidine composition to reactor 3

### DETAILED DESCRIPTION OF THE INVENTION

Guanidine produced for use as a fuel is not 100% pure guanidine. Accordingly, the term “guanidine” as used herein is intended to denote any one of the components of guanidine resulting from commercial production via a method such as that described by Kenneth J. Shaver in U.S. Pat. No. 3,108,999. These components can include free-base guanidine, urea, ammonium carbamate, ammonium carbonate, ammonium bicarbonate, ammonium formate, ammonium acetate, biuret, melamine, guanidine carbonate, guanidine hydroxide, guanylurea, or a mixture of two or more of these components. The reaction of guanidine with water can be described by the following stoichiometric equation:

CN₃H₅(s)+2H₂O(l)→3NH₃(g)+CO₂(g)ΔH=+96.3 kJ/mol (1)

This reaction is accomplished in two steps. In the first step a molecule of guanidine reacts with water to form a molecule of ammonia and a molecule of urea. The urea molecule immediately reacts with water to form two molecules of ammonia and one molecule of carbon dioxide. The immediate reaction of the urea produced by the decomposition of guanidine is a significant advantage because it allows effective solution concentrations of urea higher than the equilibrium solubility of urea in water Reaction (1) is preferably carried out at a temperature ranging between about 50° C. and about 240° C. and a pressure ranging between about 1 ambient atmosphere and about 50 standard atmospheres. The heat required for reaction (1), which is endothermic, may be obtained from waste heat generated by an engine or fuel cell in which ammonia, or hydrogen is combusted or oxidized. This heat source may be supplemented with an additional energy source, particularly to initiate reaction (1). A preferred additional energy source is a battery that may be part of the apparatus of the invention and that serves as a source of electricity.

The weight of guanidine in the composition may range between about 10% and about 90% of the composition. In one embodiment, the composition can be stored as a solid and transferred to the reaction chamber by melting at temperatures about 50° C. Pure guanidine melts at 50° C. Forming a mixture with other components such as urea will lower the melting point, in one exemplary embodiment, the composition contains guanidine and urea in a eutectic which melts, with constant composition, at a temperature below 50° C. The heat required to melt the composition may be obtained by the heat generated by an engine or fuel cell. This heat source may be supplemented by electrical resistance heating where the heating elements are inside or surrounding the container holding the composition. In an embodiment shown in FIG. 1, the guanidine composition 13 is encapsulated by a layer of material 14 that is more stable than the guanidine composition and prevents the reaction of the guanidine composition with moisture and carbon dioxide in the air. In addition, the encapsulated guanidine composition can be in the form of small granules that can be easily poured into storage containers at retail sites or in the apparatus. The encapsulating layer consists of substances that can be readily decomposed into ammonia and other gaseous products at the operating conditions used to react the guanidine composition with water to form ammonia. In another embodiment, the guanidine is dissolved in a solvent, such as water or ethanol and transferred to the reaction chamber as a solution. Guanidine is infinitely soluble in water, methanol, or ethanol, allowing use of compositions with high concentrations of guanidine which have low freezing points. The concentration of water may be chosen to exactly satisfy the stoichiometric requirements of equation (1). When ethanol is used as the solvent, the ethanol vapor exits the reaction chamber along with the ammonia and serves to enhance the combustion qualities of the resulting mixture. A distinct advantage of use of solid guanidine is the simplicity of handling a solid fuel together with the ability to precisely meter the guanidine and water into the reaction chamber in a manner to minimize the presence of excess water. Excess water will dilute the concentration of ammonia resulting from reaction (1) and can prevent proper ignition of the fuel. Use of water captured from the exhaust of an engine or fuel cell reduces the need to carry water and improves the specific energy of the fuel.

The composition may contain a component selected from the group consisting of a combustible fuel, a combustion enhancer, and a combination thereof. In the first and second embodiment of the method of the invention, the combustible fuel can be present in an amount which can be combusted to generate a sufficient amount of heat to initiate the reaction of the composition with water to form ammonia. Alternatively, the engine can begin operation on stored ammonia, and the fuel cell can begin operation on stored hydrogen until the heat produced by the engine or fuel cell is adequate to increase the reaction rate of the composition with water. A compression-ignition engine can operate initially using injection of the guanidine composition when the ethanol or methanol concentration of the composition exceeds about 30%. In the third embodiment of the method of the invention, the heat needed to start the reaction can be supplied electrically using a connection to the local electric utility lines.

The oxidation of hydrogen to form water may be performed by combusting the hydrogen in an engine. Similarly, the oxidation of ammonia to form nitrogen and water may be performed by combusting the ammonia in an engine. Alternatively, the ammonia and hydrogen may be oxidized in an ammonia fuel cell and a hydrogen fuel cell, respectively. Examples of fuel cells include high temperature fuel cells such as solid oxide fuels cell and molten carbonate fuel cells, and relatively lower temperature fuel cells such as alkaline-fuel cells, PEM fuel cells and phosphoric acid fuel cells.

The combustion of ammonia to form water and nitrogen may also lead to the formation of nitrogen oxides which have the general formula NₓO_{y}., such as, for example, NO or NO₂. Elevated levels of the nitrogen oxides are obtained depending upon reaction conditions. The nitrogen oxides are pollutants and their emission is regulated. An advantage of using ammonia as the fuel in the present invention is that pollution caused by the nitrogen oxides may be abated or eliminated by reaction of unburned ammonia exiting the combustion chamber with the nitrogen oxides in a post combustion reactor. The unburned ammonia acts as a reducing agent to convert nitrogen oxides into molecular nitrogen.

The use of ammonia and the method of reducing nitrogen oxides has been described in several patents as a way to reduce NₓO_{y} in both mobile and stationary combustion sources. Representative patents include U.S. Pat. Nos. 5,399,325, 6,403,046, 6,354,079, 6,312,650, 6,182,443, 6,146,605, 5,832,720, 5,746,144, 5,399,326, 5,281,403 and 5,240,689.

While urea and ammonia have been used as stationary source pollutant removers, these compounds have not been used for mobile applications, possibly because most consumers would not notice any change in the performance of the vehicle in the absence of the pollutant remover and would therefore forget to refill the tank containing the pollutant remover. Accordingly, manufacturers are looking for a solution that does not require a second fill-up in addition to the one required for the fuel. Currently, the solutions to this problem are to include three way catalysts as additives to the fuel or to feed small amounts of gasoline into an exhaust reactor.

The use of compositions containing guanidine provides a simple and efficient solution to the problem of pollution caused by nitrogen oxides. Because guanidine produces ammonia which acts as both the fuel and the NOx reducing agent, an apparatus that uses guanidine as the fuel also provides for its own pollution abatement. As long as there is fuel to run a vehicle there will be some unburned ammonia present in the exhaust to remove the NOₓ produced by the combustion. The air/ammonia ratio of the fuel mixture can be adjusted to supply the minimum amount of unburned ammonia in the exhaust needed to completely decompose the nitrogen oxides. Accordingly, consumers would only require one fill-up, since the same substance would serve both purposes.

The composition may also include a combustion enhancer. Preferred combustion enhancers include ammonium nitrate, guanidine nitrate, nitroguanidine, ammonia, hydrazine, and certain water soluble compounds which can be made from renewable sources or waste, such as isopropanol, ethanol and methanol. In addition to favoring combustion, such combustion enhancers may also help decrease the freezing point of the fuel when supplied in liquid form.

Several embodiments can be used to generate energy from the composition containing guanidine when reacted with water according to the method of the invention. In a first embodiment, the guanidine reacts with water to form ammonia, according to equation (1). The ammonia formed from the reaction of guanidine with water is then oxidized to form water and nitrogen generating energy. The oxidation of ammonia may be performed by combusting the ammonia in an engine. The engine may be an engine having a compression ratio at least similar to compression ratios ordinarily used in the art, such as a compression ratio of 9:1, or a compression ratio higher than compression ratios ordinarily used in the art, such as a compression ratio of 30:1 or greater, or a compression ratio ranging between 9:1 and 30:1. Alternatively, the ammonia may be heated in an ammonia fuel cell. The first embodiment of the method of the invention may further include the step of reacting a sufficient amount of the unburned ammonia in the combustion exhaust with nitrogen oxides formed from the combustion of ammonia to reduce the nitrogen oxides.

In a second embodiment of the method of the invention, the guanidine in the composition reacts with water to form ammonia, according to equation (1). The ammonia formed from the reaction of guanidine with water is then ‘reformed’ or converted into nitrogen and hydrogen. The hydrogen formed from the conversion of ammonia is then oxidized to form water generating energy. The oxidation of hydrogen may be performed by combusting the hydrogen in an engine. Alternatively, the hydrogen may be oxidized in a hydrogen fuel cell. The amount of energy required to bring the fuel cell to operating temperature for this reaction can be provided by an electric heater or by the oxidation of a small amount of hydrogen in situ.

In a third embodiment of the method of the invention, the guanidine in the composition reacts with water to form ammonia, according to equation (1). The ammonia formed from the reaction of guanidine with water is then ‘reformed’ or converted into nitrogen and hydrogen. The hydrogen is then stored in a container until being dispensed into the fuel tank of a vehicle using hydrogen as a fuel. The safety of using the solid, relatively non-flammable, guanidine composition as the means for effectively storing hydrogen and producing hydrogen on-demand with only a small amount of hydrogen in the apparatus at any time, will allow this apparatus to provide a safe source of hydrogen at numerous locations within cities and along highways as refueling stations for hydrogen-powered vehicles.

In the second and third embodiments of the method of the invention, the hydrogen formed from “reforming” ammonia can contain carbon monoxide, if the carbon dioxide product from equation (1) is not removed prior to the “reforming”. Even if the carbon dioxide is removed, the “reformed” mixture will contain traces of ammonia. Both carbon monoxide and ammonia are detrimental to the performance of many hydrogen fuel cells. These deleterious contaminants can be removed from the hydrogen by passing the “reformed” gas through a semi-permeable membrane that passes hydrogen but not other matter. This semi-permeable membrane can be a thin film of Pd metal, Pt metal, or a dense random packing of hard spheres type amorphous metal composed of mischmetal and at least one transition metal as described by Van Vechten in the U.S. patent application “DRPHS-Type Amorphous Metal Proton Transfer Membrane for Fuel Cells” filed 11 Mar. 2003 by James A. Van Vechten claiming priority to Provisional Patent Application 60/363,520 of 28 Mar. 2002.

The decomposition of guanidine according to equation (1) can be accomplished in the presence of a catalyst which increases the rate of decomposition. The catalyst may be a metal oxide together with barium hydroxide. In an exemplary embodiment, the catalyst is barium hydroxide together with an oxide of iron, nickel, vanadium or zinc. Catalysts can decrease in effectiveness due to contamination or structural changes during use. Accordingly, the catalyst in the reaction chamber can be contained in a replaceable cartridge which can be removed from the reaction chamber and replaced with a cartridge containing fresh catalyst. The removed cartridge can be reactivated or recycled.

The use of an enzyme allows the reaction of guanidine with water in equation (1) to proceed at lower temperatures than the reaction in the absence of the enzyme. The temperature of the enzyme-catalyzed reaction may range between room temperature and a temperature at which the half life of the enzyme is less than 1 minute. The enzyme may be provided in a tank equipped with a filter which prevents the enzyme from leaving but which is permeable to the gases formed. Alternatively, the enzyme may be immobilized on a substrate. Suitable substrates include ion exchange resins, ceramics, and polymeric materials. The substrate may be in the form of a sheet or beads.

In an exemplary embodiment, the enzymes capable of catalyzing the reaction of guanidine with water to form ammonia are arginase and urease. The arginase catalyzes the reaction of guanidine with water to form ammonia and urea. The urease catalyzes the reaction of urea with water to form ammonia and carbon dioxide. These enzymes catalyze the reaction at a temperature which is preferably between about 0° C. and about 60° C., more preferably about 60° C. The heat required to maintain the process at this temperature is available from proton exchange membrane (PEM) fuel cell stacks which may be easily integrated into the apparatus of the invention to provide a low temperature energy sink.

Enzymes can decrease in effectiveness due to contamination, structural changes, or denaturing of the enzyme during use. Accordingly, the enzyme in the reaction chamber can be contained in a replaceable cartridge which can be removed from the reaction chamber and replaced with a cartridge containing fresh enzyme. The removed cartridge can be reactivated or recycled.

Concern over the effects of rising carbon dioxide concentrations in the atmosphere have led to proposals to limit the emissions of carbon dioxide from vehicles and stationary energy sources. Even though the reaction of guanidine compositions with water only releases an amount of carbon dioxide equal to the amount used during the production of the guanidine composition, it may be advantageous to store the carbon dioxide produced according to equation (1), so that use of guanidine fuel together with storage of carbon dioxide will result in a net decrease of the amount of carbon dioxide released to the atmosphere. The carbon dioxide can be stored under pressure in a container or can be stored under pressure as a solution in a container holding water. The carbon dioxide gas or solution could then be transferred at fueling stations for other uses or sequestered in geologic formations. The ability to store carbon dioxide produced from guanidine compositions is feasible because the carbon to hydrogen ratio of 1:9 for guanidine is higher than for any other non-hydrogen alternative fuel.

A new alternative fuel will be successful only if it can be produced in an efficient and environmentally acceptable manner without the consumption of expensive or scarce raw materials. Accordingly, it is another object of the invention to provide a method for providing a guanidine containing composition. The method in one embodiment comprising: (a) using a source of energy to separate hydrogen from water; (b) reacting the hydrogen formed in step (a) with nitrogen to form ammonia; (c) reacting a portion of the ammonia formed in step (b) with carbon dioxide to form urea; (d) reacting the urea formed in step (c) with a portion of the ammonia formed in step (b) to form a composition containing guanidine. In one exemplary embodiment, the source of the energy in step (a) is renewable energy from wind power. The potential for wind power is extremely high in some remote regions such as the Aleutian Islands or Patagonia. The potential for harnessing the renewable energy in such remote locations has not been realized because of the lack of efficient means for storing or distributing that energy. Production of guanidine via the method of this invention provides a means to harness and store the electricity produced from renewable energy sources such as electricity generated from wind, electricity generated from ocean waves, electricity generated from hydroelectric facilities, electricity generated from solar energy, electricity generated from agricultural waste combustion, electricity generated from forestry waste combustion, electricity generated from municipal waste combustion, and electricity generated from the tidal flow of ocean water. In addition to renewable energy sources, guanidine containing compositions can also be effectively produced using electricity produced by conventional means for which the level of generating capacity exceeds the current demand. This is a form of load-leveling whereby generating plants can operate at a constant level with excess electrical energy being converted into guanidine containing compositions.

The carbon dioxide needed for the production of compositions containing guanidine can be supplied from several sources including carbon dioxide extracted from the air, carbon dioxide extracted from products created by the combustion of fossil fuels, carbon dioxide extracted from products created by the combustion of agricultural waste, carbon dioxide extracted from products created by the combustion of forestry waste, carbon dioxide extracted from products created by the combustion of municipal waste, and a combination thereof. In one exemplary embodiment, the carbon dioxide is extracted from the exhaust from a combustion process. As a result of the combustion, the exhaust is depleted of oxygen. Following the extraction of carbon dioxide, the remaining gas is primarily nitrogen which can be used as the source of nitrogen needed for production of ammonia. The nitrogen can also be supplied from conventional sources using fractional distillation of liquefied air or pressure swing absorption of air.

In addition to producing hydrogen by electrolysis for production of guanidine, use of brine or other salts in the electrolysis process can produce sodium and chlorine-containing compounds which are in high demand. The production of the chlorine-containing compounds will provide an additional source of profit and encourage the establishment of guanidine producing facilities.

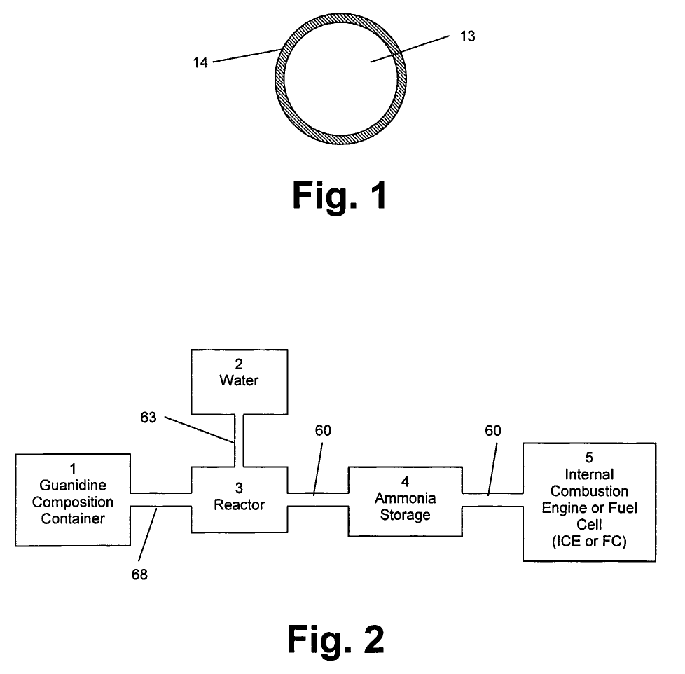

The invention is also directed to an apparatus for generating energy from guanidine compositions. In one embodiment of the invention, shown in FIG. 2, the apparatus includes a container such as tank 1 for providing the composition, and a container such as tank 2 for providing water. The composition is delivered from tank 1 to a container such as reactor 3 for reacting the guanidine composition with water, supplied from tank 2, to form ammonia. The reactor 3 for reacting the guanidine composition with water may be a container for providing a catalyst capable of catalyzing the reaction of the guanidine composition with water or an enzyme capable of catalyzing the reaction of the guanidine composition with water. The apparatus may also include a container such as buffer tank 4 for storing the ammonia produced by the reactor. The buffer tank 4 may be a container for containing an amount of ammonia which can be combusted to generate a sufficient amount of heat to initiate the reaction of the guanidine with water, preferably to instantly initiate the reaction of the guanidine composition with water. The buffer tank 4 also compensates for the difference between the production rate of ammonia from the guanidine composition and the consumption rate of ammonia. The ammonia produced from the reaction of the guanidine composition with water is delivered from the buffer tank 4 to a container such as chamber 5 for oxidizing ammonia to form water and nitrogen generating energy.

In one exemplary embodiment, chamber 5 is an ammonia fuel cell.

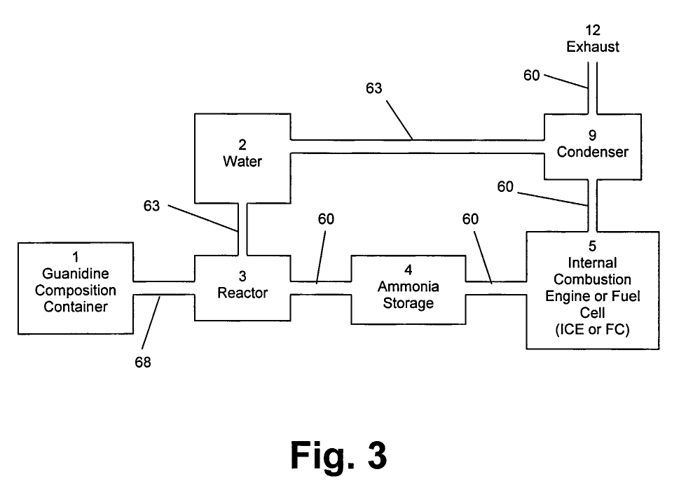

In another exemplary embodiment, shown in FIG. 3, the chamber 5 is an engine or ammonia fuel cell and the ammonia is oxidized by combusting it in the engine or oxidizing it in the fuel cell. In this exemplary embodiment, the apparatus may also include a container such container 9, connected to the engine or fuel cell for providing water, condensed from the exhaust, which is delivered to tank 2, thereby providing a portion of the water needed to react with the guanidine composition.

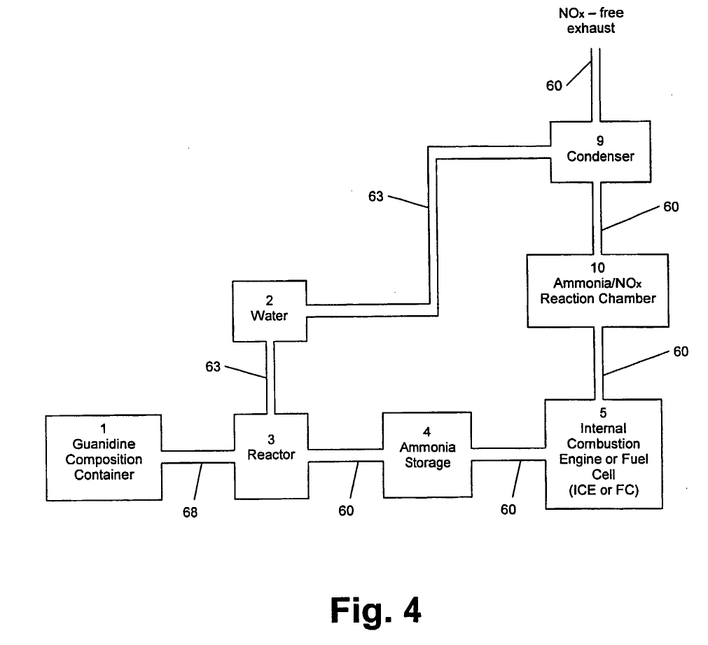

In another exemplary embodiment, shown in FIG. 4, the chamber 5 is an engine and the ammonia is oxidized by combusting it in the engine. In this exemplary embodiment, the apparatus may also include a container such container 10, connected to the engine or fuel cell serving as a reaction chamber where the unburned ammonia in the exhaust combines with and eliminates the NOx.

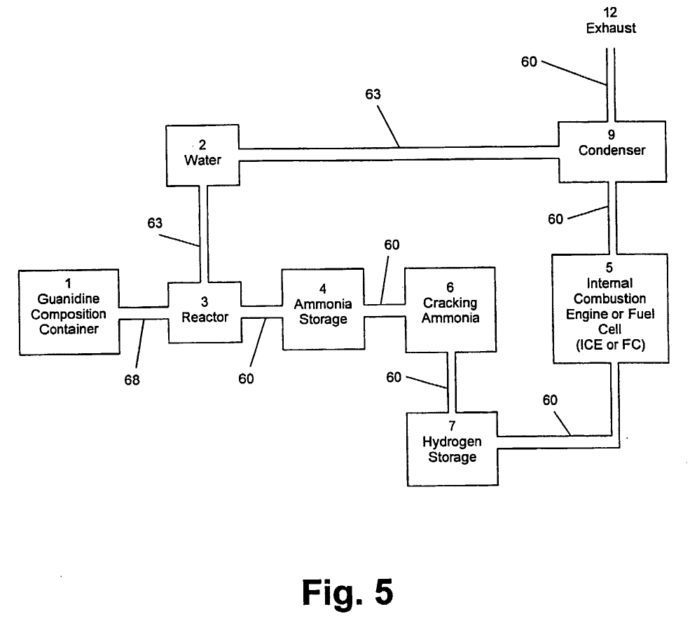

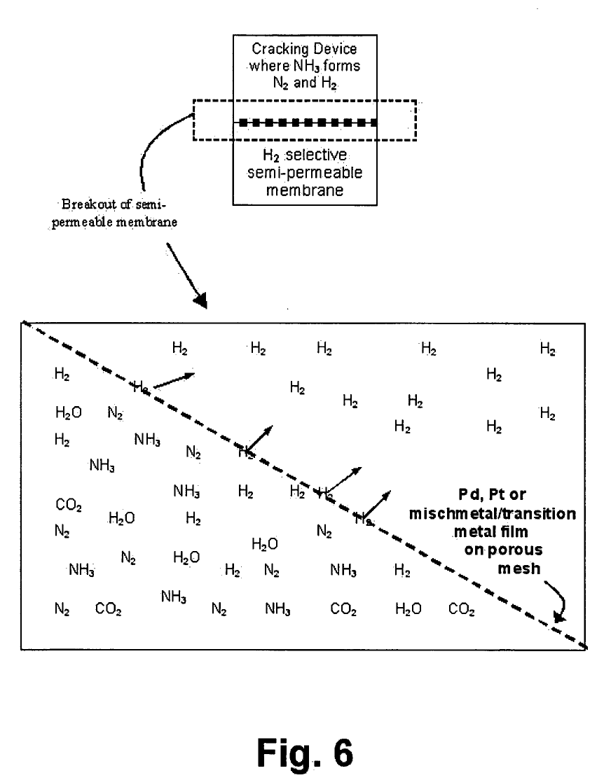

In another embodiment of the apparatus of the invention, shown in FIG. 5, the apparatus includes a container such as tank 1 for providing the composition containing guanidine and a container such as tank 2 for providing water. The composition and water are delivered to a container such as first reactor 3 for reacting the composition with water to form ammonia and carbon dioxide. The first reactor 3 for reacting the composition with water may be a container for providing a catalyst capable of catalyzing the reaction of the composition containing guanidine with water or an enzyme capable of catalyzing the reaction of the composition containing guanidine with water. The apparatus may also include a container such as buffer tank 4 for containing ammonia which is connected to the first reactor 3. The buffer tank 4 may be a container for providing an amount of ammonia which can be combusted to generate a sufficient amount of heat to initiate the reaction of the urea with water, preferably to instantly initiate the reaction of the urea with water. The ammonia produced from the reaction of the composition containing guanidine with water is delivered from the first reactor 3 to a container such as buffer tank 4 and then to the second reactor 6 for converting the ammonia into nitrogen and hydrogen. The hydrogen formed from the conversion of ammonia is delivered from the second reactor 6 to a container such as chamber 7 which serves as a buffer tank for hydrogen storage. The buffer tank 7 compensates for the difference between the production rate of hydrogen from the reformation of ammonia and the consumption rate of hydrogen. As needed, hydrogen is then delivered from chamber 7 to chamber 5 for oxidizing the hydrogen to form water generating energy. In one exemplary embodiment, chamber 5 is a hydrogen fuel cell. In another exemplary embodiment, the chamber 5 is an engine and the hydrogen is oxidized by combusting it in the engine. The apparatus may also include a container 9 for condensing a portion of the exhausted water which is delivered to tank 2, thereby providing a portion of the water needed to react with the guanidine composition. As shown in FIG. 6, The reactor 6 may include a semi-permeable membrane for separation of the hydrogen from ammonia and other products of the cracking process. FIG. 6 shows an embodiment of the second reactor 6 wherein the hydrogen is separated from ammonia and other products of the cracking process by a semi-membrane comprised of a Pd, Pt, or a dense random packing of hard spheres type amorphous metal composed of mischmetal and at least one transition metal deposited on a porous mesh.

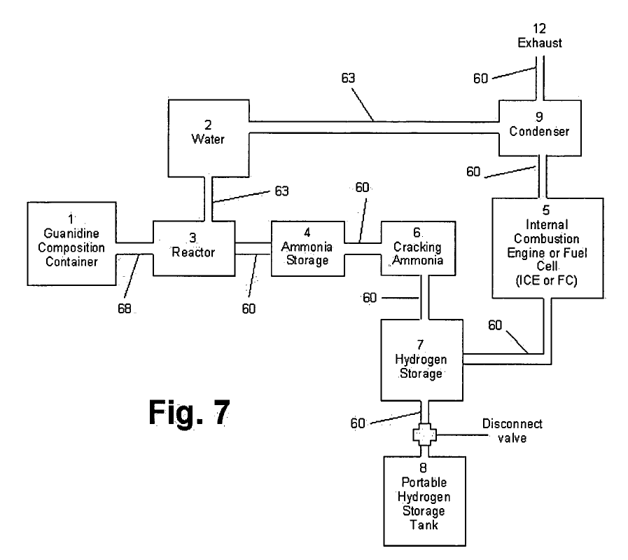

In another embodiment of the apparatus of the invention, shown in FIG. 7, the apparatus includes a container such as tank 1 for providing the composition containing guanidine and a container such as tank 2 for providing water. The composition and water are delivered to a container such as first reactor 3 for reacting the composition with water to form ammonia and carbon dioxide. The first reactor 3 for reacting the composition with water may be a container for providing a catalyst capable of catalyzing the reaction of the composition containing guanidine with water or an enzyme capable of catalyzing the reaction of the composition containing guanidine with water. The apparatus may also include a container such as buffer tank 4 for containing ammonia which is connected to the first reactor 3. The buffer tank 4 may be a container for providing an amount of ammonia which can be combusted to generate a sufficient amount of heat to initiate the reaction of the urea with water, preferably to instantly initiate the reaction of the urea with water. The ammonia produced from the reaction of the composition containing guanidine with water is delivered from the first reactor 3 to a container such as buffer tank 4 and then to the second reactor 6 for converting the ammonia into nitrogen and hydrogen. The hydrogen formed from the conversion of ammonia is delivered from the second reactor 6 to a container such as chamber 7 which serves as a buffer tank for hydrogen storage. The buffer tank 7 compensates for the difference between the production rate of hydrogen from the reformation of ammonia and the consumption rate of hydrogen. Hydrogen can be delivered from storage chamber 7 to a portable hydrogen storage tank 8 which is temporarily connected to the tank 7 for purposes of receiving hydrogen. Tank 7 can be the fuel storage tank of a hydrogen-powered vehicle. The apparatus in this embodiment can be a self-contained hydrogen fueling station that generates a portion of the power needed to operate the fueling station from a hydrogen powered engine or fuel cell 5. As needed, hydrogen is then delivered from chamber 7 to chamber 5 for oxidizing the hydrogen to form water generating energy. The apparatus may also include a container 9 for condensing a portion of the exhausted water which is delivered to tank 2, thereby providing a portion of the water needed to react with the guanidine composition

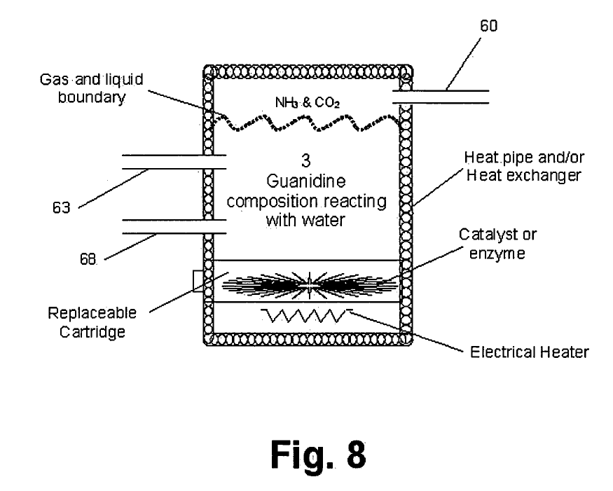

FIG. 8 shows an exemplary embodiment of the reactor 3 shown in FIGS. 2-5 and FIG. 7 for the reaction of guanidine composition with water. The replaceable cartridge containing either the catalyst for the reaction of the guanidine composition with water or the enzyme for the reaction of the guanidine composition with water can be removed and replaced to supply fresh catalyst or enzyme. An array of heat pipes surrounds the reactor, transferring heat from the engine or fuel cell to the reactor. A electrical heater provides the heat necessary to start the endothermic reaction (1).

The embodiments of FIGS. 2-5 include means 60 for delivering gaseous substances including ammonia, hydrogen, and nitrogen oxides, as well as means for delivering molten composition, water, or solutions such as the solution containing guanidine and water. Means for delivering cold solutions may include, for example, conveying means such as plastic tubing and glass manifolds. Means for delivering hot solutions and molten compositions may include conveying means such as, for example, steel or stainless steel tubes. Means 60 for delivering gaseous substances may include conveying means such as, for example, steel or stainless steel tubes, pumping means such as pumps, or a combination thereof. The pumping means may be used for pumping the gaseous substances to the extent needed to overcome pressure differentials. The embodiments of FIGS. 2-5 also include means 68 for delivering the composition comprising guanidine to the container in which the guanidine in the composition is reacted.

The apparatus of the present invention is also flexible enough to provide fuel for both combustion engines and fuel cells. Accordingly, there is no infrastructure barrier to the conversion to fuel cells.

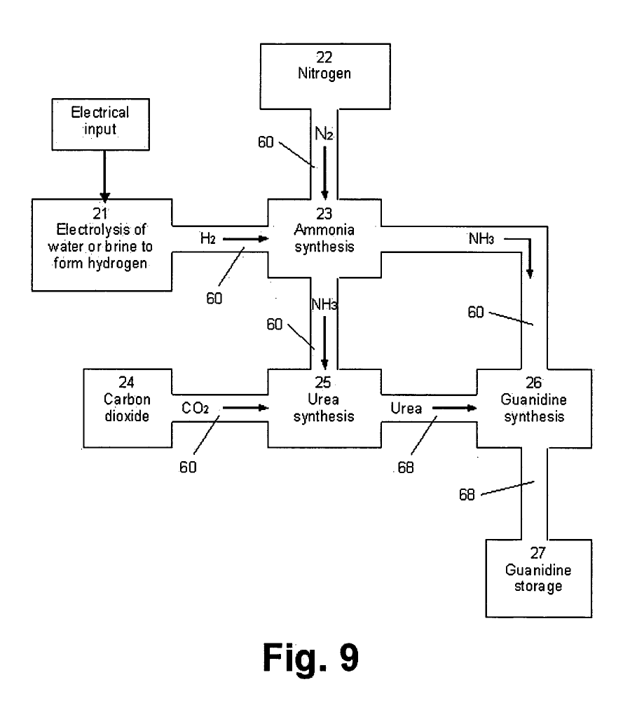

The invention is also directed to an apparatus for generating guanidine compositions from energy. In one preferred embodiment of the invention shown in FIG. 9 electrical energy from a sustainable source (wind power, tidal power, wave power) is supplied to a container where electrolysis of water or brine occurs in container 21 creating hydrogen. The hydrogen combines in reactor 23 together with nitrogen provided from container 22 to form ammonia via the Haber process. The ammonia is transferred to the urea synthesis container 25 where the ammonia is combined with carbon dioxide from container 24 to form urea. The urea is transferred to the guanidine synthesis reactor 26 where the urea is combined with additional ammonia from container 23 to form the guanidine composition. The guanidine composition is then stored in container 27.

Guanidine is produced from the reaction of ammonia gas with urea, and urea is produced from the reaction of ammonia with carbon dioxide. When reacted in the presence of water, guanidine generates ammonia and carbon dioxide. Ammonia is a known fuel which can run internal and external combustion engines. The ammonia can also be decomposed to give its constituent elements nitrogen and hydrogen. The hydrogen may then be used in an engine or in a hydrogen fuel cell. As a hydrogen source, urea contains 8.47% by weight of hydrogen. However, upon hydrolysis with two molecules of water, one molecule of guanidine produces three molecules of ammonia, with four of the hydrogen atoms coming from the water molecule. Accordingly, the guanidine-water system can theoretically yield up to 9.47% of hydrogen where the weight of all the water needed is considered in the calculation. In the case that all the water needed is captured from the exhaust and is not included in the weight calculation, guanidine can theoretically yield up to (9/59)=15.25% hydrogen by weight. The density of guanidine is approximately 1.3 kg/liter. When using water captured from the exhaust, one liter of guanidine can supply 198 grams of hydrogen. This figure of 198 grams of hydrogen per liter is extremely favorable when considering all the positive safety features of a solid guanidine-based fuel compared to cryogenic liquid hydrogen, as well as the fact that the bulk of a cryogenic tank has an unfavorable effect on the energy density value of cryogenic liquid hydrogen. The density of liquid hydrogen is 79 grams of hydrogen per liter so pure guanidine, using water captured from the exhaust, stores effectively 2.5 times more hydrogen per liter than liquid hydrogen, without any consideration of the bulk of the cryogenic tank which would reduce the amount of liquid hydrogen that could be stored in a given volume.

The present invention also has significant advantages over fossil fuels and many alternative fuels in terms of safety. The present invention gives ammonia on demand, so that at any given time the amount of ammonia present is very low and does not pose any safety concern. The guanidine composition has low toxicity and can be stored in plastic containers.

In one embodiment of the invention, as discussed above, ammonia generated from the reaction of guanidine with water is oxidized or combusted to produce energy. While pure ammonia has also been proposed as a fuel, the use of pure ammonia has several drawbacks. Ammonia is a gas at room temperature and requires high pressure to liquefy. It is a corrosive substance which in large quantities may cause respiratory problems and even death. While it is possible to dissolve ammonia in water to produce a fuel, the resulting fuel has a pH of over 11 and a strong ammonia odor. Finally, ammonia has a relatively low energy density compared to gasoline and other alternative fuels. The use of guanidine as a source of ammonia in the present invention maintains only small quantities of ammonia in the device at any time. This removes most of the problems associated with storing large quantities of ammonia in a device.

The present invention is also environmentally advantageous. Although carbon dioxide is one of the products of the hydrolysis of guanidine in reaction (1), an equal amount of carbon dioxide is removed from the atmosphere to make guanidine in the first place. Accordingly, the production of ammonia according to the invention does not entail any net contribution to greenhouse gas emissions. The other reactant required for the preparation of guanidine is ammonia, which in turn requires a hydrogen source for its manufacture. Most of the hydrogen used for this purpose is supplied by the process of steam reforming of natural gas, which releases carbon dioxide into the atmosphere. However, hydrogen can also be prepared by an environmentally friendly method such as the electrolysis of water, where the electricity needed for the process is supplied by nuclear, hydroelectric, solar, tidal or wind power, none of which is a greenhouse gas emitting process. In this case, the required amount of carbon dioxide may be obtained by sequestering carbon dioxide from flue gases, or from the atmosphere, which contains 370 ppm CO₂, as needed. Accordingly, it is possible using existing technology to prepare guanidine in a manner that is economical and environmentally friendly compared to fossil fuels.

1. A method for generating energy from a composition containing guanidine, the method comprising: (a) reacting the composition with water to form ammonia and carbon dioxide; and (b) oxidizing the ammonia formed in step (a) to form water and nitrogen generating energy.

2. The method of claim 1, wherein the weight of guanidine in the composition is equal to about 10% to about 100% of the weight of the composition.

3. The method of claim 1, wherein a portion of the water formed in step (b) is reacted with the composition in step (a) to form ammonia.

4. The method of claim 1, wherein the composition reacts with water in step (a) at a temperature ranging between about 50° C. and about 240° C. and a pressure ranging between about 1 ambient atmosphere and 50 standard atmospheres.

5. The method of claim 4, wherein the method comprises mixing or contacting the composition with a catalyst capable of catalyzing the reaction of the composition with water in step (a).

6. The method of claim 5, wherein the catalyst is an oxide of a metal, wherein the metal is selected from the group consisting of iron, nickel, vanadium, and zinc.

7. The method of claim 5, wherein the catalyst is a composition of barium hydroxide and an oxide of a metal, wherein the metal is selected from the group consisting of iron, nickel, vanadium and zinc.

8. The method of claim 1, wherein the composition further comprises a component selected from the group consisting of a combustible fuel, a combustion enhancer, ammonia, ammonium bicarbonate, ammonium carbonate, ammonium hydroxide, aminoguanidine, aminoguanidine bicarbonate, aminoguanidine carbonate, aminoguanidine hydroxide, diaminoguanidine, diaminoguanidine bicarbonate, diaminoguanidine carbonate, diaminoguanidine hydroxide, triaminoguanidine, triaminoguanidine bicarbonate, triaminoguanidine carbonate, triaminoguanidine hydroxide, guanidine, guanidine bicarbonate, guanidine carbonate, guanidine hydroxide, guanylurea, urea, water, and a combination thereof.

9. The method of claim 8, wherein the component is a combustible fuel present in an amount which can be combusted to generate a sufficient amount of heat to initiate a reaction of guanidine with water.

10. The method of claim 1, wherein the composition is encapsulated in a layer, wherein the material of the encapsulating layer is selected from the group consisting of urea, guanidine, guanidine carbonate, guanidine bicarbonate, guanylurea, ammonium carbonate, ammonium bicarbonate and a combination thereof.

11. The method of claim 1, wherein the method comprises mixing or contacting the composition with an enzyme capable of catalyzing the reaction of the composition with water.

12. The method of claim 11, wherein the enzyme is capable of catalyzing the reaction of the composition with water at a temperature ranging between room temperature and a temperature at which the half life of the enzyme is less than 1 minute.

13. The method of claim 11, wherein the enzyme is selected from the group consisting of arginase, urease, and a combination thereof.

14. The method of claim 1, wherein step (b) comprises burning the ammonia formed in step (a).

15. The method of claim 14, wherein nitrogen oxides are formed in step (b) and the method further comprises reacting a portion of the unburned ammonia from the exhaust of combustion step (b) with the nitrogen oxides to reduce the nitrogen oxides and the unburned ammonia.

16. The method of claim 14, wherein the ammonia is combusted in an internal combustion engine having a compression ratio selected from the group consisting of a compression ratio between 9:1 and 30:1 and a compression ratio greater than 30:1.

17. The method of claim 1, wherein at least a fraction of the carbon dioxide is stored under pressure to serve one or more purposes chosen from prevention of release to the atmosphere, provision of a reactant in a chemical process, provision of inert gas to extinguish fire, provision of feed stock for dry ice, preserving food, provision of compressed gas to power a pneumatic device and a combination thereof.

18. The method of claim 1, wherein a portion of the water produced in step (b) is retained to aid in the retention of the carbon dioxide under pressure.

19. A method for generating energy from a composition containing guanidine comprising: (a) reacting the composition with water to form ammonia and carbon dioxide; (b) converting the ammonia formed in step (a) to nitrogen and hydrogen; and (c) oxidizing the hydrogen formed in step (b) to form water generating energy.

20. The method of claim 19, wherein the weight of the weight of guanidine in the composition is equal to about 10% to about 100% of the weight of the composition.

21. The method of claim 19, wherein the composition reacts with water in step (a) at a temperature ranging between about 50° C. and about 240° C. and a pressure ranging between about 1 ambient atmosphere and 50 standard atmospheres.

22. The method of claim 19, wherein a portion of the water exhausted by a fuel cell or by an internal or external combustion engine is reacted with the composition to form ammonia in step (a).

23. The method of claim 21, wherein the method comprises mixing or contacting the composition with a catalyst capable of catalyzing the reaction of the composition with water in step (a).

24. The method of claim 23, wherein the catalyst is an oxide of a metal, wherein the metal is selected from the group consisting of iron, nickel, vanadium and zinc.

25. The method of claim 23, wherein the catalyst is a composition of barium hydroxide and an oxide of a metal, wherein the metal is selected from the group consisting of iron, nickel, vanadium and zinc.

26. The method of claim 19, wherein the composition further comprises a component selected from the group consisting of a combustible fuel, a combustion enhancer, ammonia, ammonium bicarbonate, ammonium carbonate, ammonium hydroxide, aminoguanidine, aminoguanidine bicarbonate, aminoguanidine carbonate, aminoguanidine hydroxide, diaminoguanidine, diaminoguanidine bicarbonate, diaminoguanidine carbonate, diaminoguanidine hydroxide, triaminoguanidine, triaminoguanidine bicarbonate, triaminoguanidine carbonate, triaminoguanidine hydroxide, guanidine, guanidine bicarbonate, guanidine carbonate, guanidine hydroxide, guanylurea, urea, water, and a combination thereof.

27. The method of claim 26, wherein the component is a combustible fuel present in an amount which can be combusted to generate a sufficient amount of heat to initiate a reaction of guanidine with water.

28. The method of claim 19, wherein the composition is encapsulated in a layer, wherein the material of the encapsulating layer is selected from the group consisting of urea, guanidine, guanidine carbonate, guanidine bicarbonate, guanylurea, ammonium carbonate, ammonium bicarbonate and a combination thereof.

29. The method of claim 19, wherein the method comprises mixing or contacting the composition with an enzyme capable of catalyzing the reaction of the composition with water.

30. The method of claim 29, wherein the enzyme is capable of catalyzing the reaction of the composition with water at a temperature ranging between room temperature and a temperature at which the half life of the enzyme is less than 1 minute.

31. The method of claim 29, wherein the enzyme is selected from the group consisting of arginase, urease, and a combination thereof.

32. The method of claim 19 wherein the hydrogen is separated by passing through a semi-permeable membrane that passes hydrogen but not other matter.

33. The method of claim 32 wherein the semi-permeable membrane is a thin film of Pd metal, Pt metal, or a dense random packing of hard spheres type amorphous metal composed of mischmetal and at least one transition metal.

34. A method for providing a guanidine containing composition, the method comprising: (a) using a source of energy to separate hydrogen from water; (b) reacting the hydrogen formed in step (a) with nitrogen to form ammonia; (c) reacting a portion of the ammonia formed in step (b) with carbon dioxide to form urea; (d) reacting the urea formed in step (c) with a portion of the ammonia formed in step (b) to form a composition containing guanidine.

35. The method of claim 34 wherein the source of energy in step (a) is selected from the group consisting of electricity generated from wind, electricity generated from ocean waves, electricity generated from hydroelectric facilities, electricity generated from solar energy, electricity generated from nuclear energy, electricity from a power grid provided as part of a load-leveling process, electricity generated from fossil fuel combustion, electricity generated from agricultural waste combustion, electricity generated from forestry waste combustion, electricity generated from municipal waste combustion, electricity generated from the tidal flow of ocean water, solar energy as used in a chemical process for the splitting of water into hydrogen and oxygen, and a combination thereof.

36. The method of claim 34 wherein method used to provide the nitrogen in step (b) is selected from the group consisting of fractional distillation of liquefied air, a pressure swing absorption process, collection of the partially oxygen-depleted exhaust from a combustion process, and a combination thereof.

37. The method of claim 34 wherein the source of carbon dioxide in step (c) is selected from the group consisting of carbon dioxide extracted from the air, carbon dioxide extracted from products created by the combustion of fossil fuels, carbon dioxide extracted from products created by the combustion of agricultural waste, carbon dioxide extracted from products created by the combustion of forestry waste, carbon dioxide extracted from products created by the combustion of municipal waste, and a combination thereof.

38. The method of claim 34, wherein the weight of guanidine in the composition formed in step (d) is equal to about 10% to about 100% of the weight of the composition.

39. The method of claim 34, wherein the composition further comprises a component selected from the group consisting of ammonia, ammonium bicarbonate, ammonium carbonate, ammonium hydroxide, aminoguanidine, aminoguanidine bicarbonate, aminoguanidine carbonate, aminoguanidine hydroxide, diaminoguanidine, diaminoguanidine bicarbonate, diaminoguanidine carbonate, diaminoguanidine hydroxide, triaminoguanidine, triaminoguanidine bicarbonate, triaminoguanidine carbonate, triaminoguanidine hydroxide, guanidine bicarbonate, guanidine carbonate, guanidine hydroxide, guanylurea, urea, water, and a combination thereof.

40. The method of claim 34, wherein the energy is used to separate hydrogen from a solution containing sodium chloride, thereby producing sodium and chlorine-containing substances.

41. An apparatus for generating energy from a composition containing guanidine, comprising: (a) a first container for providing the composition; (b) a second container providing water; (c) a third container for reacting the composition with water to form ammonia and carbon dioxide, wherein the third container is connected to the first container by means for delivering the composition from the first container to the third container and the third container is connected to the second container by means for delivering water from the second container to the third container; (d) a fourth container for storing ammonia, wherein the fourth container is connected to the third container by means for delivering ammonia from the third container to the fourth container; and (e) a fifth container for oxidizing ammonia to form water and nitrogen generating energy, wherein the fifth container is connected to the fourth container by means for delivering ammonia from the fourth container to the fifth container.

42. The apparatus of claim 41, wherein the fifth container for oxidizing ammonia is an engine for combusting ammonia.

43. The apparatus of claim 42, wherein nitrogen oxides are formed from the combustion of ammonia, and the apparatus further comprises (a) a sixth container providing a catalyst for the decomposition of unburned ammonia and nitrogen oxides; and (b) a means for delivering the nitrogen oxides and unburned ammonia from the engine to the sixth container.

44. The apparatus of claim 41, wherein the fourth container is a container for providing an amount of ammonia which can be combusted to generate a sufficient amount of heat to initiate the reaction of the composition with water to form ammonia.

45. The apparatus of claim 41, wherein the third container for reacting the composition with water to form ammonia is a container for providing a catalyst capable of catalyzing the reaction of the composition with water.

46. The apparatus of claim 41, wherein the third container for reacting the composition with water to form ammonia is a container for providing an enzyme capable of catalyzing the reaction of the composition with water.

47. The apparatus of claim 45, wherein the third container is fitted with a replaceable cartridge containing the catalyst.

48. The apparatus of claim 46, wherein the third container is fitted with a replaceable cartridge containing the enzyme.

49. The apparatus of claim 41, wherein the fifth container for oxidizing ammonia is an ammonia fuel cell.

50. The apparatus of claim 41, wherein heat is transferred from the fifth container to the third container to accelerate the reaction of the composition with water.

51. The apparatus of claim 41, wherein the first container is removable from the apparatus for purposes of transferring the composition to the apparatus.

52. An apparatus for generating energy from a composition containing guanidine comprising: (a) a first container for providing the composition; (b) a second container providing water; (c) a third container for reacting the composition with water to form ammonia and carbon dioxide, wherein the third container is connected to the first container by means for delivering the composition from the first container to the third container and the third container is connected to the second container by means for delivering water from the second container to the third container; (d) a fourth container for storing ammonia, wherein the fourth container is connected to the third container by means for delivering ammonia from the third container to the fourth container; and (e) a fifth container for converting ammonia to nitrogen and hydrogen, wherein the fifth container is connected to the fourth container by means for delivering ammonia from the fourth container to the fifth container; (e) a sixth container for storing hydrogen, wherein the sixth container is connected to the fifth container by means for delivering hydrogen from the fifth container to the sixth container; and (f) a seventh container for oxidizing hydrogen to form water generating energy, wherein the seventh container is connected to the sixth container by means for delivering hydrogen from the sixth container to the seventh container.

53. The apparatus of claim 52, wherein the seventh container for oxidizing hydrogen is an engine for combusting hydrogen.

54. The apparatus of claim 52, wherein the fourth container is a container for providing an amount of ammonia which can be combusted to generate a sufficient amount of heat to initiate the reaction of the composition with water to form ammonia.

55. The apparatus of claim 52, wherein the third container for reacting the composition with water to form ammonia is a container for providing a catalyst capable of catalyzing the reaction of the composition with water.

56. The apparatus of claim 52, wherein the third container for reacting the composition with water to form ammonia is a container for providing an enzyme capable of catalyzing the reaction of the composition with water.

57. The apparatus of claim 55, wherein the third container is fitted with a replaceable cartridge containing the catalyst.

58. The apparatus of claim 56, wherein the third container is fitted with a replaceable cartridge containing the enzyme

59. The apparatus of claim 52, wherein the seventh container for oxidizing hydrogen is a hydrogen fuel cell.

60. The apparatus of claim 52, wherein heat is transferred from the seventh container to the third container to accelerate the reaction of the composition with water.

61. The apparatus of claim 52, wherein the first container is removable from the apparatus for purposes of transferring the composition to the apparatus.

62. An apparatus for generating energy from a composition containing guanidine comprising: (a) a first container for providing the composition; (b) a second container providing water; (c) a third container for reacting the composition with water to form ammonia, wherein the third container is connected to the first container by means for delivering the composition from the first container to the third container and the third container is connected to the second container by means for delivering water from the second container to the third container; (d) a fourth container for storing ammonia, wherein the fourth container is connected to the third container by means for delivering ammonia from the third container to the fourth container; and (e) a fifth container for converting ammonia to nitrogen and hydrogen, wherein the fifth container is connected to the fourth container by means for delivering ammonia from the fourth container to the fifth container; (e) a sixth container for storing hydrogen, wherein the sixth container is connected to the fifth container by means for delivering hydrogen from the fifth container to the sixth container; and (f) a means for delivering hydrogen from the sixth container to a hydrogen storage tank in a vehicle which uses hydrogen as a fuel to generate energy.

63. The apparatus of claim 62, wherein the apparatus is mobile

64. The apparatus of claim 63, wherein a portion of the electrical energy required for operation of the apparatus and a portion of the heat required for the endothermic decomposition of the guanidine composition is supplied by a fuel cell.

65. An apparatus for providing a composition containing guanidine comprising: (a) a first container for separating hydrogen from water using energy; (b) a second container for providing nitrogen; (c) a third container for reacting hydrogen with nitrogen to form ammonia; wherein the third container is connected to the first container by means for delivering hydrogen from the first container to the second container and the third container is connected to the second container by means for delivering nitrogen from the second container to the third container; (d) a fourth container providing carbon dioxide; (e) a fifth container for reacting ammonia with carbon dioxide to form urea; wherein the fifth container is connected to the third container by means for delivering ammonia from the third container to the fifth container and the fifth container is connected to the fourth container by means for delivering the carbon dioxide from the fourth container to the fifth container; (f) a sixth container for reacting urea with ammonia to form a composition containing guanidine; wherein the sixth container is connected to the third container by means for delivering ammonia from the third container to the sixth container, and the sixth container is connected to the fifth container by means for delivering urea from the fifth container to the sixth container; (g) a seventh container for storing the guanidine composition; wherein the seventh container is connected to the sixth by means for delivering the guanidine composition from the sixth container to the seventh container.
